(19) United States
(12) Patent Application Publication
(10) Pub. No.: US 2019/0211297 A1
(43) Pub. Date: Jul. 11, 2019

(54) LARGE CIRCULATING FLUIDIZED BED CELL BIOREACTOR AND METHOD FOR CULTURING ANIMAL CELLS

(71) Applicant: Ni HUI, Beijing (CN)

(72) Inventors: Ni HUI, Beijing (CN); Shiyao HUI, Beijing (CN); Ii LI, Beijing (CN)

(21) Appl. No.: 16/326,826

(22) PCT Filed: Oct. 14, 2016
(86) PCT No.: PCT/CN2016/102076

§ 371 Date: Feb. 20, 2019

(30) Foreign Application Priority Data
Sep. 27, 2016 (CN) CN201610857483.6

(51) Int. Cl. C12M 1/12; C12M 1/00; C12N 5/00; B01J 8/00; B01J 8/02

(52) U.S. Cl. CPC C12M 25/20; C12M 29/06; C12N 5/0075; B01J 8/0285; B01J 8/003; B01J 8/0228; C12M 29/02

(57) ABSTRACT

A large circulating fluidized bed cell bioreactor and a method for culturing animal cells. The reactor comprises an agitator tank body (01), an agitator tank base (50), a water inlet silicone hose (06), a backflow silicone hose (08), a cell culture tank (10), a culture tank base (40), a backflow pipe (15), a water inlet pipe (12), an agitator (20), a reactor tray (30), and a tube support plate (60). By means of cooperation among the above components, defects and shortcomings of fluidized bed cell bioreactors in the prior art including proneness to microbial contamination, a low level of dissolved oxygen, and a low working volume, are effectively resolved, advantageous in the popularization and application of this technology.

### TECHNICAL FIELD

The disclosure relates to the technical field of cell bioreactors, in particular to a large circulating fluidized bed cell reactor and a method for culturing animal cells, and more particularly to a large circulating fluidized bed cell reactor for culturing animal cells and a method for large-scale culturing of animal cells.

### BACKGROUND ART

At present, there is a very wide market application prospect of expressing and producing various monoclonal antibodies for diagnosing and treating, vaccines, growth factors and other biologically active proteins by culturing animal cells in vitro. Therefore, cell bioreactors for large-scale and high-density culturing of animal cells have become a critical apparatus in the biotechnology pharmaceutical industry. It determines product cost and quality, production scale and product type of biopharmaceutical enterprises to a great extent.

A fluidized bed cell bioreactor includes a reaction tank body, a temperature controlling portion, a ventilating portion, a feeding inlet and a discharge outlet, a measuring system, a control system, and other auxiliary systems.

The existing fluidized bed cell reactors are all in a structure of mechanical stirring devices inserted into the tank body. The structure and the sealing elements of the existing fluidized bed cell reactors often have problems of leakage and microbial contamination. This leads to a reduction in safety and reliability, and a high probability of bacterial infection. Further, the culture solution has a low level of dissolved oxygen, which also cannot meet the requirements for high-density culturing of cells. Still Further, the reactor has a small working volume, and it is difficult to implement a truly large-scale culturing even with a perfusion culture process.

### SUMMARY

An object of the disclosure is to provide a large circulating fluidized bed cell bioreactor and a method for culturing animal cells, so as to address defects and shortcomings of the fluidized bed cell bioreactors existing in the prior art including proneness to microbial contamination, a low level of dissolved oxygen, and a small working volume, etc.

In order to solve the aforementioned technical problems, an aspect of the disclosure provides a large circulating fluidized bed cell bioreactor, comprising an agitator tank body, an agitator tank base, a water inlet silicone hose, a backflow silicone hose, a cell culture tank, a culture tank base, a backflow pipe, a water inlet pipe, an agitator, a reactor tray, and a tube support plate; the agitator tank body is provided in the agitator tank base, the agitator tank base is connected to an agitator tray of the agitator, the agitator is mounted on the reactor tray; the agitator tank body has a central hopper, and a fluid-guiding pipe is disposed at an upper portion of the agitator tank body, an inlet of the fluid-guiding pipe is connected to an inner wall of the agitator tank body, an outlet of the fluid-guiding pipe enters the central hopper along an tangential direction of an upper portion of the central hopper; the central hopper is located at a central position of the agitator tank body, with a lower end thereof protruding a bottom portion of the agitator tank body; the cell culture tank is provided in the culture tank base, the culture tank base is mounted on the reactor tray; a bottom portion and an upper portion of the cell culture tank are correspondingly connected with the water inlet pipe and the backflow pipe respectively; the water inlet pipe, the water inlet silicone hose, and a bottom portion of the central hopper are sequentially connected, the backflow pipe, the backflow silicone hose, and a lower end of a side-position backflow outlet at the bottom portion of the agitator tank body are sequentially connected; and the tube support plate supports the water inlet pipe and the backflow pipe at the same time.

Optionally, the agitator tank body is in a shape of a truncated cone, with a half cone angle of 25 degrees to 55 degrees (for example, which may be 30 degrees, 35 degrees, 40 degrees, 50 degrees, etc.), preferably 30 degrees to 42 degrees; and the agitator tank body has a tank cover with various water inlets.

Further, the cell culture tank has a bottom truncated cone portion and an upper truncated cone portion, has a first cylindrical portion between the bottom truncated cone portion and the upper truncated cone portion, and has a second cylindrical portion between the upper truncated cone portion and a top portion of the cell culture tank.

Further, a small head end at the top portion of the cell culture tank is connected to an inlet end of the backflow pipe, an outlet end of the backflow pipe is connected to a lower end of the backflow silicone hose; and the bottom truncated cone portion of the cell culture tank is connected to a lower end of the water inlet pipe along its own tangential direction, and an upper end of the water inlet pipe is connected to a lower end of the water inlet silicone hose.

Preferably, the cell culture tank has a truncated cone-shaped filter screen inside, and the truncated cone-shaped filter screen is connected to an inner wall of the second cylindrical portion at an upper portion of the cell culture tank, and a cone apex of the truncated cone-shaped filter screen is made upward.

Optionally, the bottom truncated cone portion and the upper truncated cone portion of the cell culture tank, and the truncated cone-shaped filter screen all have a half cone angle of 25 degrees to 60 degrees (for example, which may be 30 degrees, 35 degrees, 40 degrees, 50 degrees, 55 degrees etc.), preferably 45 degrees to 60 degrees.

Further, a top portion of the cell culture tank has a tank cover with various inlets and outlets, and the tank cover is connected with a flange at an edge of a top end of the cell culture tank, and sealed; and an inner wall of the cell culture tank base is adhered with a heating rubber plate.

Preferably, an area of a cross section of the upper truncated cone portion is enlarged gradually from bottom to top.

Another aspect of the disclosure provides a method for culturing animal cells using the large circulating fluidized bed cell bioreactor provided by the disclosure and comprising the following steps:

after adding culture solution to a static liquid level, the culture solution is heated to 35.5-37° C.; an agitator is actuated (rotating at a rotating rate ranging from 70-110 rpm), disinfected microcarriers are added to the culture solution in proportion, and then active animal cells are inoculated into the microcarriers; the culture solution at an upper portion of the agitator tank body dissolves oxygen in an oxygen-dissolved area in an upper space of the agitator tank body, in the fluid-guiding pipe and in the central hopper, and the culture solution after dissolving oxygen enters the water inlet silicone hose and the water inlet pipe under an action of an agitating force; then enters the cell culture tank along a tangential direction of the bottom truncated cone portion of the cell culture tank, starts to flow upward in a spiral manner until flowing to a lower edge of the truncated cone-shaped filter screen, delivering nutrients and dissolved oxygen to active cells in the microcarriers in this region; and after this supply process is completed, the culture solution flows through the truncated cone-shaped filter screen to a top portion of the culture tank and flows into the backflow pipe, then flows back into the agitator tank body through the backflow silicone hose and a side-position backflow outlet at a bottom portion of the agitator tank body, flows upward in a spiral manner and re-enters an inlet of the fluid-guiding pipe, and a cycle is completed; and then a next cycle starts after replenishing oxygen and nutrients.

Further, in the process where the cell culture tank delivers nutrients and oxygen to the active animal cells, the microcarriers with the active animal cells are in a region between the bottom truncated cone portion of the cell culture tank and the truncated cone-shaped filter screen at an upper portion; and when the culture solution containing the dissolved oxygen and nutrients flows upward, nutrients and dissolved oxygen are delivered to the active animal cells in the microcarriers.

Further, the microcarriers are macroporous microcarriers or porous microcarriers having a weight and a shape, each of the microcarriers having a relatively downward settling rate in the culture solution; wherein, when an upward flow rate of the culture solution and a downward settling rate of the microcarriers are relatively balanced, the microcarriers are balanced and do not move, instead of settling downward; when the upward flow rate of the culture solution is higher than an equilibrium value, the microcarriers flow upward together with the culture solution; when the upward flow rate of the culture solution is lower than the equilibrium value, the microcarriers settle downward; and by structural characteristics of the upper truncated cone portion with an gradually enlarged area of cross section, the upward flow rate of the culture solution in this section is thus reduced, which is favorable for the microcarriers to settle downward, such that a rapid and reliable separation of the microcarriers from the culture solution is completed at this part.

Preferably, a diameter of a pore of the truncated cone-shaped filter screen is smaller than a diameter of the microcarrier.

A large circulating fluidized bed cell bioreactor and a method for culturing animal cells provided by the disclosure can bring about at least one of the following beneficial effects.

1. With respect to requirements of large-scale and high-density culturing of animal cells, the current various cell reactors mainly have problems including: a problem about reliability in terms of bacterial infection, and an occasional leakage due to a poor sealing reliability of a bearing section of the stirring blade inserted into the tank, which reduces the reliability and results in failure of cell culturing. The large circulating fluidized bed cell bioreactor provided by the disclosure has a brand new structure and principle without a stirring device inserted into the tank, and has good overall sealing performance and quite high safety and reliability.

2. With respect to the sensitivity of animal cells to the shearing force, in the current bioreactor, the fluid shearing force and bubble rupture caused by the stirring blade and by bubbling cause damage even death to cells, which results in failure in culturing. Therefore, there is no stirring blade in the tank body of the large circulating fluidized bed cell bioreactor provided by the disclosure, and an oxygen-dissolved area where a lot of bubbles occur is in the agitator tank body, and the region where the cells live is in the cell culture tank, and they are not the same region, and thus there is a very low degree of damage to cells, moreover, the large circulating fluidized bed cell bioreactor provided by the disclosure cultures cells using macroporous microcarriers within which the cells are further protected by the hard shell of the macroporous microcarriers.

3. With respect to the issue of the level of dissolved oxygen in the culture solution, the current cell reactor addresses the oxygen-dissolved issue by means of the stirring of the stirring blade and the bubbling, but the level of dissolved oxygen is generally low, and especially in the process of large-scale and high-density culturing of animal cells, with low level of dissolved oxygen, it is always incapable to meet the requirement of respiration of animal cells, and it is difficult to culture animal cells with high density, at high quality and high efficiency. However, the large circulating fluidized bed cell bioreactor provided by the disclosure utilizes a circumferential oscillation of the agitator, where a large amount of water droplets and waves are generated within the agitator tank, which increases an area where the surface of the culture solution and mixing gas in the upper portion of the agitator tank are contacted and dissolved with each other, and enlarges the parameter of the gas-liquid phase specific surface area in the oscillating state, so that a high level of dissolved oxygen is reached, and it has been found in the experiment by measurement that the level of dissolved oxygen in the oscillating state can be much higher than that in the conventional reactor, which fully meets the requirement of culturing cells with high density and at high quality.

4. With respect to the issue of the working volume of the cell reactor, in general, in the process of large-scale culturing of animal cells with cell bioreactor, with other conditions unchanged, the larger the working volume is, the higher the efficiency is, and the relatively lower the cost will be; because the product cultures animal cells using macroporous microcarriers and using a highly efficient perfusion process, its working volume is calculated as follows: an actual working volume is a daily perfusion amount multiplied by the number of days of perfusion; generally, the daily perfusion amount is 0.5-2 times of the actual working volume of the culture tank, and if this coefficient is set as 1, that is, the daily perfusion amount is the actual volume of the cell culture tank of the product, the number of days of perfusion can generally up to about 30 days; if the cell culture tank of the large circulating fluidized bed cell bioreactor provided by the disclosure has a volume of 500 liters, then the actual working volume is 15,000 liters that equals to 500 liters/day multiplied by 30 days; and with the large circulating fluidized bed cell bioreactor provided by the disclosure, the following has been experimentally proven: in case of ensuring the vertically upward flow rate of the culture solution in the cell culture tank no less than 60 cm/min, the agitator tank drives the cell culture tank at a ratio of the volume of 1 to 10, that is, when the agitator tank has a volume of 200 liters, it can drive a cell reaction tank with a volume of 2000 liters, and under this condition, the actual working volume, which the cells are cultured using the perfusion process, is 60,000 liters which equals to 2000 liters/day multiplied by 30 days, it is a relatively large batch both in the biopharmaceutical industry and the biological product industry, and such a large working volume is rarely used in actual production, which is another big advantage of the large circulating fluidized bed cell bioreactor provided by the disclosure that the actual working volume is able to be very large.

### DESCRIPTION OF THE DRAWINGS

Drawings required for use in the description of specific embodiments or the prior art will be introduced briefly below in order to explain the technical solutions of the specific embodiments of the disclosure or of the prior art more clearly, it will be apparent that the drawings in the description below are merely some embodiments of the disclosure, and those ordinarily skilled in the art can also obtain, according to these drawings, other drawings without inventive efforts.

FIG. 1 is a schematic diagram of the structural principle of a large circulating fluidized bed cell bioreactor provided by an embodiment of the disclosure;

FIG. 2 is a top structural schematic view of the large circulating fluidized bed cell bioreactor shown in FIG. 1;

FIG. 3 is a front structural schematic view of an agitator tank body;

FIG. 4 is a side structural schematic view of an agitator tank body; and

FIG. 5 is a top structural schematic view of an agitator tank body.

### DETAILED DESCRIPTION OF THE EMBODIMENTS

The technical solutions of the disclosure will be described clearly and completely below with reference to the drawings. It is apparent that the embodiments described are some embodiments of the disclosure, but not all of the embodiments. Based on the embodiments of the disclosure, all the other embodiments, obtained by those ordinarily skilled in the art without inventive efforts, will fall within the scope of protection of the disclosure.

In the description of the disclosure, it should be indicated that orientations or positional relations indicated by terms such as “center”, “up”, “down”, “left”, “right”, “vertical”, “horizontal”, “inside”, and “outside” are based on the orientations or positional relations as shown in the figures, only for facilitating description of the disclosure and simplifying the description, rather than indicating or implying that the referred devices or elements must be in a particular orientation or constructed or operated in the particular orientation, and therefore they should not be construed as limiting the disclosure. In addition, terms such as “first”, “second”, and “third” are used only for the purpose of description, and should not be construed as indicating or implying relative importance.

In the description of the disclosure, it should be indicated that unless otherwise expressly specified or defined, terms “mount”, “join”, and “connect” should be understood broadly, and for example, a connection may be a fixed connection, or a detachable connection, or an integrated connection; may be a mechanical connection or an electric connection; may be a direct joining, or an indirect joining via an intermediate medium, or may be an internal communication between two elements. The specific meanings of the above-mentioned terms in the disclosure could be understood by those ordinarily skilled in the art according to specific situations.

The disclosure will be further described in detail below with reference to specific embodiments in conjunction with drawings.

### Embodiment 1

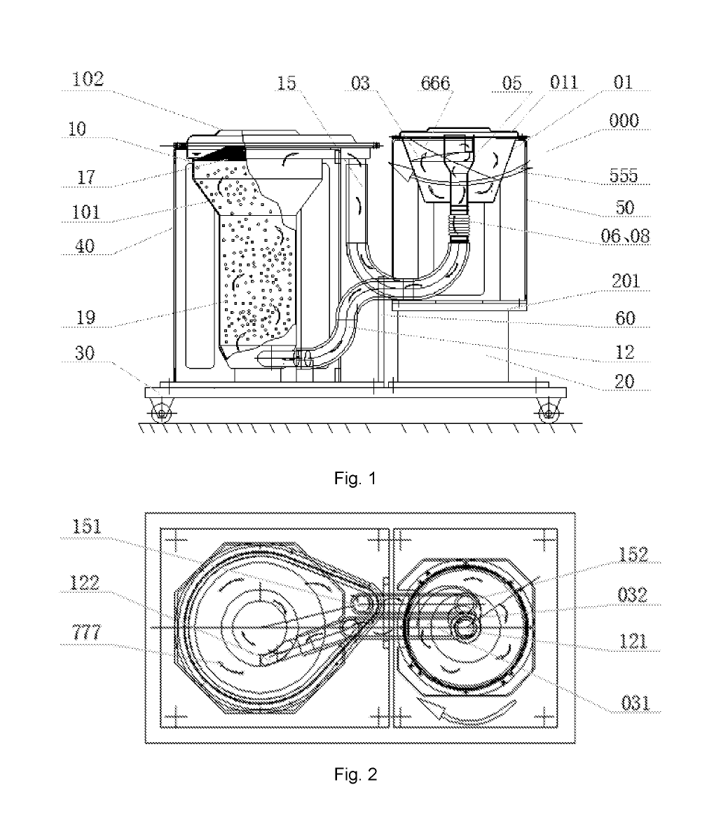

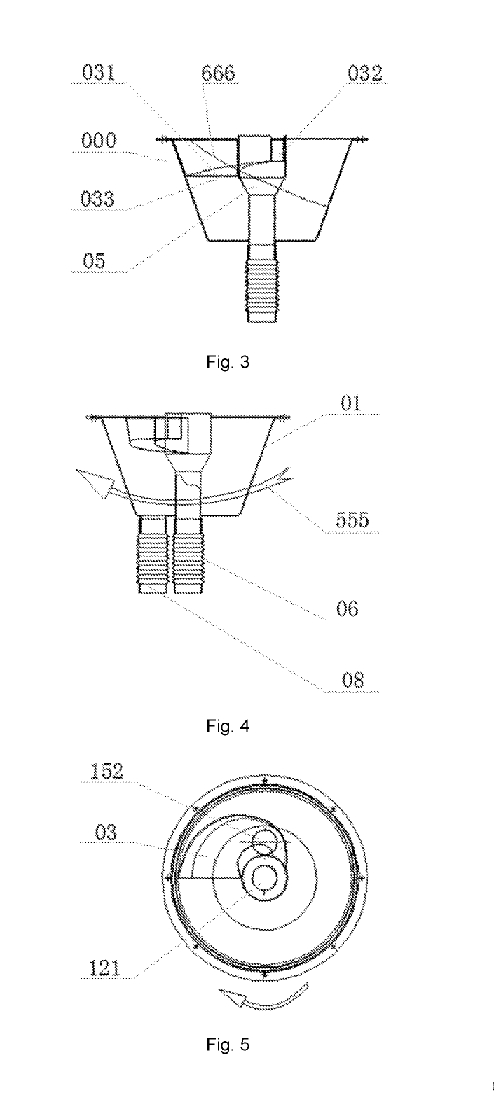

As shown in FIGS. 1 to 5, the Embodiment 1 provides a large circulating fluidized bed cell bioreactor, comprising an agitator tank body 01, an agitator tank base 50, a water inlet silicone hose 06, a backflow silicone hose 08, a cell culture tank 10, a culture tank base 40, a backflow pipe 15, a water inlet pipe 12, an agitator 20, a reactor tray 30, and a tube support plate 60; the agitator tank body 01 is provided in the agitator tank base 50, the agitator tank base 50 is connected to an agitator tray 201 of the agitator 20, the agitator 20 is mounted on the reactor tray 30; the agitator tank body 01 has a central hopper 05, and a fluid-guiding pipe 03 is disposed at an upper portion of the agitator tank body 01, an inlet 031 of the fluid-guiding pipe connects an inner wall of the agitator tank body 01, an outlet 032 of the fluid-guiding pipe enters the central hopper 05 along an tangential direction of an upper portion of the central hopper 05; the central hopper 05 is located at a central position of the agitator tank body 01, with a lower end thereof protruding a bottom portion of the agitator tank body 01; the cell culture tank 10 is provided in the culture tank base 40, the culture tank base 40 is mounted on the reactor tray 30; a bottom portion and an upper portion of the cell culture tank 10 are correspondingly connected with the water inlet pipe 12 and the backflow pipe 15 respectively; the water inlet pipe 12, the water inlet silicone hose 06, and a bottom portion of the central hopper 05 are sequentially connected, the backflow pipe 15, the backflow silicone hose 08, and a side-position backflow outlet 152 at a bottom portion of the agitator tank body 01 are sequentially connected; and the tube support plate 60 supports the water inlet pipe 12 and the backflow pipe 15 at the same time.

### Embodiment 2

As shown in FIGS. 1 to 5, in the Embodiment 2, the agitator tank body 01 and the auxiliary structure are described in detail based on the above-mentioned Embodiment 1. Of course, the technical solution in Embodiment 1 also falls within the scope of protection of Embodiment 2.

In one example, specifically, the agitator tank body 01 is in a shape of a truncated cone, with a half cone angle of 25 degrees; and the agitator tank body 01 has a tank cover with a water inlet. That is, the agitator tank body 01 is provided thereon with an agitator tank cover 011 having an air inlet, an air outlet, a liquid inlet and a liquid outlet, and the agitator tank body 01 and attached portions are mounted in the agitator tank base 50, a ring flange at a bottom portion of the agitator tank base 50 and an agitator tray 201 are connected by a bolt, and the chassis of the agitator 20 is connected with the reactor tray 30 by a bolt to fix the agitator 20 to the reactor tray 30.

Optionally, in another example, the agitator tank body 01 is in a shape of a truncated cone, with a half cone angle of 55 degrees; and the agitator tank body 01 has a tank cover with a water inlet. That is, the agitator tank body 01 is provided thereon with an agitator tank cover 011 having an air inlet, an air outlet, a liquid inlet and a liquid outlet, and the agitator tank body 01 and attached portions are mounted in the agitator tank base 50, a ring flange at a bottom portion of the agitator tank base 50 and the agitator tray 201 are connected by a bolt, and the chassis of the agitator 20 is connected with the reactor tray 30 by a bolt to fix the agitator 20 to the reactor tray 30.

According to the application, the truncated cone-shaped agitator tank body 01 generally has a half cone angle of 25 degrees to 55 degrees, preferably 30 degrees to 42 degrees which are better, depending on the specific circumstances.

Preferably, an inner wall of the agitator tank base 50 is provided with an electrically heating rubber plate which enables the thermal insulation of the agitator tank body 01.

More specifically, an inlet side of the fluid-guiding pipe 03 is connected onto a conical surface at an inner side of the agitator tank body 01, an outlet of the fluid-guiding pipe 03 is connected to an upper portion of the central hopper 05, and when the agitator tank is agitated in a clockwise agitating direction 555, the culture solution also undergoes a circumferential oscillating flow. At this time, the culture solution level is changed from a static liquid level 000 to a dynamic liquid level 666, and simultaneously also in a clockwise rotational motion. The culture solution in the circumferential oscillating motion flows in from the inlet of the fluid-guiding pipe 03, flows out from the outlet of the fluid-guiding pipe 03, and enters the upper portion of the central hopper 05 from the tangential direction. It flows downward in a clockwise direction in a swirling manner, then flows out at a bottom outlet of the central hopper 05, enters the water inlet silicone hose 06 then flows into an inlet 121 of the water inlet pipe, and then flows downward to an outlet 122 of the water inlet pipe, enters into the cell culture tank 10, flows upward in a spiral manner from the bottom truncated cone portion, flows through the first cylindrical portion of the cell culture tank 10, enters the upper truncated cone portion 101, and then flows to the second cylindrical portion to further reach a top portion of the cell culture tank 10. It flows downward at the small head end at a top portion into the inlet 151 of the backflow pipe, and then flows downward and then returns upwardly to the backflow silicone hose 08, and flows back to the agitator tank body 01 through the backflow outlet 152. With the circumferential oscillating motion of the agitator tank body 01, the culture solution returning to the agitator tank body 01 continues to flow upward clockwise in a spiral manner, and enters again the inlet 031 of the fluid-guiding pipe, and with such a reciprocating, forming a circulating flow of culture solution between the agitator tank body 01 and the cell culture tank 10.

Here, in the agitator tank body 01, there is neither microcarrier nor cell. After agitator 20 is actuated, a circumferential oscillating motion is generated in the culture solution in the agitator tank, which drives the culture solution in the entire incubator to flow circularly. The agitator tank is the source power. In the agitator tank body 01, a mixing gas entering the tank through an air inlet on the tank cover is mixed, in gas-liquid phase, with the surface of the water droplets and the waves of the culture solution at the upper portion of the entire tank body, in the fluid-guiding pipe 03 and in the central hopper 05, dissolving the mixing gas entering the culture solution, and the non-dissolved residual gas rises to the liquid level and then escapes, and at the same time, the culture solution with the mixing gas dissolved flows into the water inlet pipe 12 and then flows into a bottom portion of the cell culture tank 10. The nutrients and dissolved oxygen carried by the culture solution are supplied to the cells in macroporous microcarriers 19.

### Embodiment 3

As shown in FIGS. 1 to 5, in the Embodiment 3, the cell culture tank 10 and the auxiliary structure are described in detail based on the above-mentioned Embodiment 1 and Embodiment 2. Of course, the technical solution in Embodiment 1 also falls within the scope of protection of Embodiment 2.

In one example, specifically, the cell culture tank 10 has a bottom truncated cone portion and an upper truncated cone portion 101, has a first cylindrical portion between the bottom truncated cone portion and the upper truncated cone portion 101, and has a second cylindrical portion between the upper truncated cone portion 101 and a top portion of the cell culture tank. Preferably, the bottom truncated cone portion and the upper truncated cone portion 101 of the cell culture tank 10, and the truncated cone-shaped filter screen 17 all have a half cone angle of 25 degrees.

In another example, optionally, the cell culture tank 10 has a bottom truncated cone portion and an upper truncated cone portion 101, and has a first cylindrical portion between the bottom truncated cone portion and the upper truncated cone portion 101, and has a second cylindrical portion between the upper truncated cone portion 101 and a top portion of the cell culture tank. Optionally, the bottom truncated cone portion and the upper truncated cone portion 101 of the cell culture tank 10, and the truncated cone-shaped filter screen 17 all have a half cone angle of 60 degrees.

Of course, according to the application, the bottom truncated cone portion and the upper truncated cone portion 101 of the cell culture tank 10, and the truncated cone-shaped filter screen 17 may have a half cone angle that may be flexibly set within the range of 25-60 degrees according to actual condition. Here, the truncated cone-shaped filter screen 17 has a cone surface with generally a half cone angle of 40 degrees to 60 degrees, preferably 45 degrees to 60 degrees which are better, depending on the specific circumstances.

The cell culture tank 10 has a truncated cone-shaped filter screen 17 inside, and the truncated cone-shaped filter screen 17 is connected to an inner wall of the second cylindrical portion at an upper portion of the cell culture tank 10, and a cone apex of the truncated cone-shaped filter screen 17 is made upward. The upper cylindrical portion in the cell culture tank 10 is provided with the truncated cone-shaped filter screen 17, a lower edge of the truncated cone-shaped filter screen 17 is connected with an inner wall of the cylindrical tank, and an upper edge is higher than the static liquid level 000, specifically 30 mm higher.

A diameter of a pore on the truncated cone-shaped filter screen 17 is smaller than a diameter of the microcarrier 19, so that the microcarrier 19 can be reliably filtered out, with only the culture solution passed. The cell culture tank 10 is filled with a mixing solution having microcarriers 19 from a bottom portion to the upper truncated cone-shaped filter screen 17, where the cells live in the microcarriers 19. Under an effect of the truncated cone-shaped filter screen 17, when the microcarriers 19 with the cells flow upward in a spiral manner with the culture solution, only the culture solution can smoothly flow through, and the microcarriers 19 are all blocked by the filter screen. Therefore, the region where the cells live is in a region from a bottom portion of the cell culture tank 10 to an underneath of the truncated cone-shaped filter screen 17.

A small head end at a top portion of the cell culture tank 10 is connected to an inlet 151 of the backflow pipe, the backflow pipe 15 resembles a U-shaped pipe which goes downward and then upward, and an outlet end of the backflow pipe 15 is connected at a lower end of the backflow silicone hose 08, and an upper end of the backflow silicone hose 08 is connected to a side-position backflow outlet 152 at a bottom portion of the agitator tank body to form a loop between the cell culture tank 10 and the agitator tank body 01. Correspondingly, the bottom truncated cone portion of the cell culture tank 10 is connected to a lower end of the water inlet pipe 12 along its own tangential direction, and an upper end of the water inlet pipe 12 is connected to a lower end of the water inlet silicone hose 06.

Moreover, a top portion of the cell culture tank has a cell culture tank cover 102 with an air inlet, an air outlet, a liquid inlet, a liquid outlet, a sampling port and various electrode sockets. The cell culture tank 10 and the attached portions are mounted in a cell culture tank base 40. The ring flange at a bottom portion of the cell culture tank base 40 is connected with the reactor tray 30 by a bolt and fixed onto the reactor tray 30. Moreover, an inner wall of the cell culture tank base 40 is further adhered with a heating rubber plate. In addition, the reactor tray 30 is welded thereon with a tube support plate 60, and a top portion of the tube support plate 60 is connected with the backflow pipe 15 and the water inlet pipe 12 to support the two water pipes and fix them.

### Embodiment 4

As shown in FIGS. 1 to 5, the Embodiment 4 further provides a method for culturing animal cells based on the above-mentioned Embodiment 1, Embodiment 2 and Embodiment 3, comprising the following steps:

In one example, after adding culture solution to a static liquid level 000, the culture solution is heated to 37° C.; an agitator 20 is actuated, specifically rotating at a rotating rate of 110 rpm, disinfected microcarriers 19 are added to the culture solution in proportion, then active animal cells are inoculated to the microcarriers 19; the culture solution at an upper portion of the agitator tank body 01 dissolves oxygen in an oxygen-dissolved area in the fluid-guiding pipe 03 and in the central hopper 05, where culture solution after dissolving oxygen enters the water inlet silicone hose 06 and the water inlet pipe 12 under an action of an agitating force, then enters the cell culture tank 10 along a tangential direction of the bottom truncated cone portion of the cell culture tank 10, starts to flow upward in a spiral manner until flowing to a lower edge of the truncated cone-shaped filter screen 17, delivering nutrients and dissolved oxygen to active cells in the microcarriers 19 in this region; and after this supply process is completed, the culture solution flows through the truncated cone-shaped filter screen 17 to a top portion of the culture tank and flows into the backflow pipe 15, then flows back to the agitator tank body 01 through the backflow silicone hose 08 and a side-position backflow outlet 152 at a bottom portion of the agitator tank body, flows upward in a spiral manner and re-enters an inlet of the fluid-guiding pipe 03, and a cycle is completed; and a next cycle starts after replenishing oxygen and nutrients.

In another example, with other conditions unchanged, the culture solution is heated to 35.5° C.; and the agitator 20 is actuated to rotate at a rotating rate of 70 rpm.

In the process where the cell culture tank 10 delivers nutrients and oxygen to the active animal cells, the microcarriers 19 with the active animal cells are in a region between the bottom truncated cone portion of the cell culture tank 10 and the truncated cone-shaped filter screen 17 at the upper portion; and when the culture solution containing the dissolved oxygen and nutrients flows upward, nutrients and dissolved oxygen are delivered to the active animal cells in the microcarriers 19.

The microcarriers 19 are macroporous microcarriers 19 or porous microcarriers 19 having a weight and a shape, each of the microcarriers 19 having a relatively downward settling rate in the culture solution; wherein, when an upward flow rate of the culture solution and a downward settling rate of the microcarriers 19 are relatively balanced, the microcarriers 19 are balanced and do not move, instead of settling downward; when an upward flow rate of the culture solution is higher than an equilibrium value, the microcarriers 19 flow upward together with the culture solution; and when the upward flow rate of culture solution is lower than the equilibrium value, the microcarriers 19 settle downward.

By structural characteristics of the upper truncated cone portion 101 with a gradually enlarged area of the cross section, the upward flow rate of the culture solution in this section is thus reduced, which is favorable for the microcarrier 19 to settle downward, such that a rapid and reliable separation of the microcarriers 19 from the culture solution is completed at this part, and a reliable basic condition is provided for the application of the perfusion culture process. The upper truncated cone portion 101 of the cell culture tank 10 has a function of decelerating the flow rate of the mixing culture solution flowing upward in a spiral manner. In this portion, since the cross-sectional area of the truncated cone portion of the culture tank becomes larger, the upward flow rate of the mixing culture solution that flows from the bottom up in a spiral manner changes inversely, that is, the upward flow rate becomes smaller. When the upward velocity value is less than the equilibrium value with respect to the settling rate of the microcarriers 19, the microcarriers 19 stop or settle vertically downward, no longer flowing upward. For this reason, in this region, the microcarriers 19 do not flow upward along with the flow, and only the culture solution itself continues to flow upward. Plus the effect of the filter screen, the culture solution and the microcarriers 19 can be completely separated reliably here. The microcarriers 19 with the cells retain always in the region from the bottom portion of the cell culture tank 10 to the lower edge of the filter screen. The culture solution flows to a top portion of the tank and flows into the backflow pipe 15, and returns to the agitator tank, and the next cycle proceeds.

Finally, it should be illustrated that the above embodiments are only used to illustrate the technical solutions of the disclosure, rather than limiting the same; although the disclosure has been described in detail with reference to the foregoing embodiments, those of ordinary skill in the art should understand that: it is still possible to modify the technical solutions described in the foregoing embodiments or equivalently replace some or all of the technical features thereof; and these modifications or replacements do not deviate the essence of the corresponding technical solutions from the scope of the technical solutions of the embodiments of the disclosure.

1. A large circulating fluidized bed cell bioreactor, comprising an agitator tank body, an agitator tank base, a water inlet silicone hose, a backflow silicone hose, a cell culture tank, a culture tank base, a backflow pipe, a water inlet pipe, an agitator, a reactor tray, and a tube support plate,
wherein the agitator tank body is provided in the agitator tank base, the agitator tank base is connected to an agitator tray of the agitator, the agitator is mounted on the reactor tray; a central hopper is provided in the agitator tank body, and a fluid-guiding pipe is disposed at an upper portion of the agitator tank body, an inlet of the fluid-guiding pipe is connected to an inner wall of the agitator tank body, an outlet of the fluid-guiding pipe enters the central hopper along an tangential direction of an upper portion of the central hopper; and the central hopper is located at a central portion of the agitator tank body, with a lower end protruding from a bottom portion of the agitator tank body; and
the cell culture tank is provided in the culture tank base, the culture tank base is mounted on the reactor tray; a bottom portion and an upper portion of the cell culture tank are connected with the water inlet pipe and the backflow pipe respectively; the water inlet pipe, the water inlet silicone hose, and a bottom portion of the central hopper are sequentially connected, the backflow pipe, the backflow silicone hose, and a side-position backflow outlet at the bottom portion of the agitator tank body are sequentially connected; and the tube support plate supports both the water inlet pipe and the backflow pipe.

2. The large circulating fluidized bed cell bioreactor according to claim 1, wherein the agitator tank body is in a shape of a truncated cone, with a half cone angle of 25 degrees to 55 degrees.

3. The large circulating fluidized bed cell bioreactor according to claim 1, wherein the agitator tank body has a tank cover with a water inlet.

4. The large circulating fluidized bed cell bioreactor according to claim 1, wherein the cell culture tank has a bottom truncated cone portion and an upper truncated cone portion, and has a first cylindrical portion between the bottom truncated cone portion and the upper truncated cone portion, and a second cylindrical portion between the upper truncated cone portion and a top portion of the cell culture tank.

5. The large circulating fluidized bed cell bioreactor according to claim 4, wherein a small head end of the top portion of the cell culture tank is connected to an inlet end of the backflow pipe, an outlet end of the backflow pipe is connected to a lower end of the backflow silicone hose; and the bottom truncated cone portion of the cell culture tank is connected to a lower end of the water inlet pipe along its own tangential direction, and an upper end of the water inlet pipe is connected to a lower end of the water inlet silicone hose.

6. The large circulating fluidized bed cell bioreactor according to claim 5, wherein a truncated cone-shaped filter screen is provided in the cell culture tank, and the truncated cone-shaped filter screen is connected to an inner wall of the second cylindrical portion at an upper portion of the cell culture tank, and the truncated cone-shaped filter screen is arranged with its cone apex upward.

7. The large circulating fluidized bed cell bioreactor according to claim 6, wherein the bottom truncated cone portion and the upper truncated cone portion of the cell culture tank, and the truncated cone-shaped filter screen all have a half cone angle of 25 degrees to 60 degrees.

8. The large circulating fluidized bed cell bioreactor according to claim 1, wherein the top portion of the cell culture tank has a tank cover with an inlet and an outlet, and the tank cover is sealingly connected with a flange at an edge of a top end of the cell culture tank.

9. The large circulating fluidized bed cell bioreactor according to claim 8, wherein an inner wall of the culture tank base is adhered with a heating rubber plate.

10. The large circulating fluidized bed cell bioreactor according to claim 1, wherein the agitator tank body is in a shape of a truncated cone, with a half cone angle of 30 degrees to 42 degrees.

11. The large circulating fluidized bed cell bioreactor according to claim 4, wherein an area of a cross section of the upper truncated cone portion is enlarged gradually from bottom to top.

12. The large circulating fluidized bed cell bioreactor according to claim 6, wherein the bottom truncated cone portion and the upper truncated cone portion of the cell culture tank, and the truncated cone-shaped filter screen all have a half cone angle of 45 degrees to 60 degrees.

13. A method for culturing animal cells, using the large circulating fluidized bed cell bioreactor according to claim 1 and comprising steps of:
heating, after adding a culture solution to a static liquid level, the culture solution to 35.5-37° C.; actuating an agitator, adding disinfected microcarriers to the culture solution in proportion, then inoculating active animal cells to the microcarriers; making the culture solution at the upper portion of the agitator tank body dissolve oxygen in an oxygen-dissolved area in an upper space of the agitator tank body, in the fluid-guiding pipe and in the central hopper, wherein the culture solution after dissolving oxygen enters the water inlet silicone hose and the water inlet pipe under an action of an agitating force; then enters the cell culture tank along a tangential direction of the bottom truncated cone portion of the cell culture tank, starts to flow upward in a spiral manner until flowing to a lower edge of a truncated cone-shaped filter screen, delivering nutrients and dissolved oxygen to active cells in the microcarriers in this region; and after this supply process is completed, the culture solution flows through the truncated cone-shaped filter screen to a top portion of the culture tank and flows into the backflow pipe, then flows back to the agitator tank body through the backflow silicone hose, flows upward in a spiral manner and re-enters the inlet of the fluid-guiding pipe, and a cycle is completed; and a next cycle starts after replenishing oxygen and nutrients.

14. The method for culturing animal cells according to claim 13, wherein in the process where the cell culture tank delivers nutrients and oxygen to the active animal cells, the microcarriers with the active animal cells are in a region between the bottom truncated cone portion of the cell culture tank and the truncated cone-shaped filter screen at an upper portion; and when the culture solution containing the dissolved oxygen and nutrients flows upward, nutrients and dissolved oxygen are delivered to the active animal cells in the microcarriers.

15. The method for culturing animal cells according to claim 13, wherein the microcarriers are macroporous microcarriers or porous microcarriers having a weight and a shape, each of the microcarriers having a relatively downward settling rate in the culture solution; wherein when an upward flow rate of the culture solution and the downward settling rate of the microcarriers are relatively balanced, the microcarriers are balanced and do not move, instead of settling downward; and
when the upward flow rate of the culture solution is higher than an equilibrium value, the microcarriers flow upward together with the culture solution; when the upward flow rate of the culture solution is lower than the equilibrium value, the microcarriers settle downward; and by means of structural characteristics of an upper truncated cone portion with a gradually enlarged area of a cross section, the upward flow rate of the culture solution in this section is thus reduced, which is favorable for the microcarriers to settle downward, such that a rapid and reliable separation of the microcarriers from the culture solution is completed at this part.

16. The method for culturing animal cells according to claim 13, wherein a diameter of a pore of the truncated cone-shaped filter screen is smaller than a diameter of each of the microcarriers.

17. The method for culturing animal cells according to claim 13, wherein the agitator tank body is in a shape of a truncated cone, with a half cone angle of 25 degrees to 55 degrees.

18. The method for culturing animal cells according to claim 13, wherein the agitator tank body has a tank cover with a water inlet.

19. The method for culturing animal cells according to claim 13, wherein the cell culture tank has a bottom truncated cone portion and an upper truncated cone portion, and has a first cylindrical portion between the bottom truncated cone portion and the upper truncated cone portion, and a second cylindrical portion between the upper truncated cone portion and a top portion of the cell culture tank.

20. The method for culturing animal cells according to claim 13, wherein the top portion of the cell culture tank has a tank cover with an inlet and an outlet, and the tank cover is sealingly connected with a flange at an edge of a top end of the cell culture tank.
